(19) United States
(12) Patent Application Publication
(10) Pub. No.: US 2006/0116579 A1
(43) Pub. Date: Jun. 1, 2006

(54) Ultrasound imaging apparatus and method thereof

(75) Inventors: Pai-Chi Li, Taipei City (TW); Sheng-Wen Huang, Taishan Township (TW)

Correspondence Address: DENNISON, SCHULTZ, DOUGHERTY & MACDONALD, 1727 KING STREET, SUITE 105, ALEXANDRIA, VA, 22314, US

(73) Assignee: Pai-Chi LI

(21) Appl. No.: 10/997,969

(22) Filed: Nov. 29, 2004

(51) Int. Cl. A61B 8/14

(52) U.S. Cl. 600/444

(57) ABSTRACT

An ultrasound imaging apparatus and method comprises a transducer array, a positioning device for the array, a reflector, a multiplexer, a transmitter/receiver, an image reconstruction and signal processing apparatus, and a monitor. The transducer array and the reflector are located on two sides of an object which are opposite to each other. Every channel of the transducer array can independently transmit and receive signal, and the reflector is used to reflect the transmitted signal from any channel. Thus, the system can collect the echoes from the reflector for all transmit/receive combinations, and then extract from the echoes the time-of-flight data and the attenuation data necessary for estimating the sound velocities and the attenuation coefficients, respectively. When estimating the sound velocities and the attenuation coefficients of different tissues in an object, the B-mode image is applied to provide the segmentation information of the object in order to improve the estimation accuracy. The invention can be used to obtain the ultrasound B-mode image, the sound velocities, and the attenuation coefficients of an object, and can be extended to obtaining other parameters, such as elastic modulus, with a single apparatus.

### BACKGROUND OF THE INVENTION

1. Field of the Invention

The present invention relates to an ultrasound imaging system, and, in particular, to an ultrasound imaging apparatus and method thereof for obtaining the ultrasound B-mode image, the sound velocities, the attenuation coefficients, and the elastic properties of an object, which can be used for tissue characterization (e.g., to detect the breast tumor).

2. Description of the Prior Art

Ultrasound computed tomography is a standard approach to reconstructing the sound velocities and the attenuation coefficients [1]˜[3]. As shown in FIG. 1, two ultrasound transducers 11 and 12 are located next to an object, and opposite to each other. Given a location in a specific angle, the transmit transducer 11 transmits an ultrasonic signal, and the receive transducer 12 receives the signal to complete a single measurement. Then the transducers 11 and 12 are moved transversely together in a short distance to next location for repeating the transmitting and receiving process to complete another measurement.

With enough times of movements, a set of measurements in this specific angle relative to the object 4 can be completed. Then the transducers 11 and 12 are rotated together to another specific angle with being kept opposite to each other, and all processes are repeated to get another set of measurements at the new angle. After the angles span 180 degrees, the sound velocities and the attenuation coefficients, as functions of the position, of the object can be estimated. Although the sound velocities and the attenuation coefficients can be obtained using the conventional imaging apparatus 1, the B-mode imaging that can be used to obtain the backscattering information is not available with this setup.

Thus, the conventional apparatus and method is improvable.

In view of the above-mentioned disadvantages, the inventors develop an ultrasound imaging apparatus and method, which improves the conventional apparatus and method.

### SUMMARY OF THE INVENTION

One aspect of the invention is to provide an ultrasound imaging apparatus and method, which uses ultrasound to obtain the B-mode image, the sound velocities, the attenuation coefficients, and the elastic properties of an object, and then differentiates tissues, e.g. tumor, in accordance with these data.

Another aspect of the invention is to provide an ultrasound imaging apparatus and method, which uses an ultrasound transducer array for transmitting and receiving ultrasound signals, without rotating the array or the object, to obtain the B-mode image, the sound velocities, and the attenuation coefficients. The invention applies the B-mode image of the object for assisting in estimating the sound velocities and the attenuation coefficients.

Another aspect of the invention is to provide an ultrasound imaging apparatus and method, which applies the information extracted from the B-mode image of the object to efficiently improve the estimation accuracy of the sound velocities and the attenuation coefficients without using other imaging apparatus.

An ultrasound imaging apparatus and method achieving the above-mentioned aspects comprises a transducer array, a positioning device for the array, a reflector, a multiplexer, a transmitter/receiver, an image reconstruction and signal processing apparatus, and a monitor. The transducer array and the reflector are located on two sides of an object. Every channel of the transducer array can independently transmit and receive signal, and the reflector is used to reflect the transmitted signal from any channel. Therefore, the system can collect the echoes from the reflector for all transmit/receive combinations, and then extract from the echoes the time-of-flight data and the attenuation data necessary for estimating the sound velocities and the attenuation coefficients, respectively. When estimating the sound velocities and the attenuation coefficients of different tissues in an object, the B-mode image is applied for providing the segmentation information of the object to improve the estimation accuracy. The invention can be used to obtain the ultrasound B-mode image, the sound velocities, and the attenuation coefficients of an object, and can be extended to obtaining other parameters, such as elastic modulus, with a single apparatus.

### BRIEF DESCRIPTION OF THE DRAWINGS

The drawings disclose an illustrative embodiment of the present invention which serves to exemplify the various advantages hereof, and are as follows:

FIG. 1 is a diagram for the setup of the conventional ultrasound computed tomography;

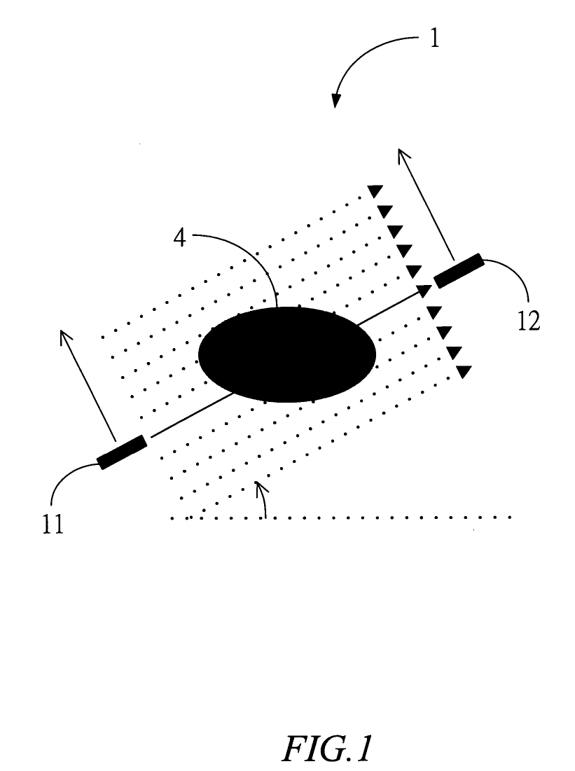

FIG. 2 is a diagram of the imaging equipment of the invention;

FIG. 3 is a schematic diagram of the imaging apparatus in the invention; and

FIG. 4 illustrates an implementation method of the invention.

### DETAILED DESCRIPTION OF THE PREFERRED EMBODIMENT

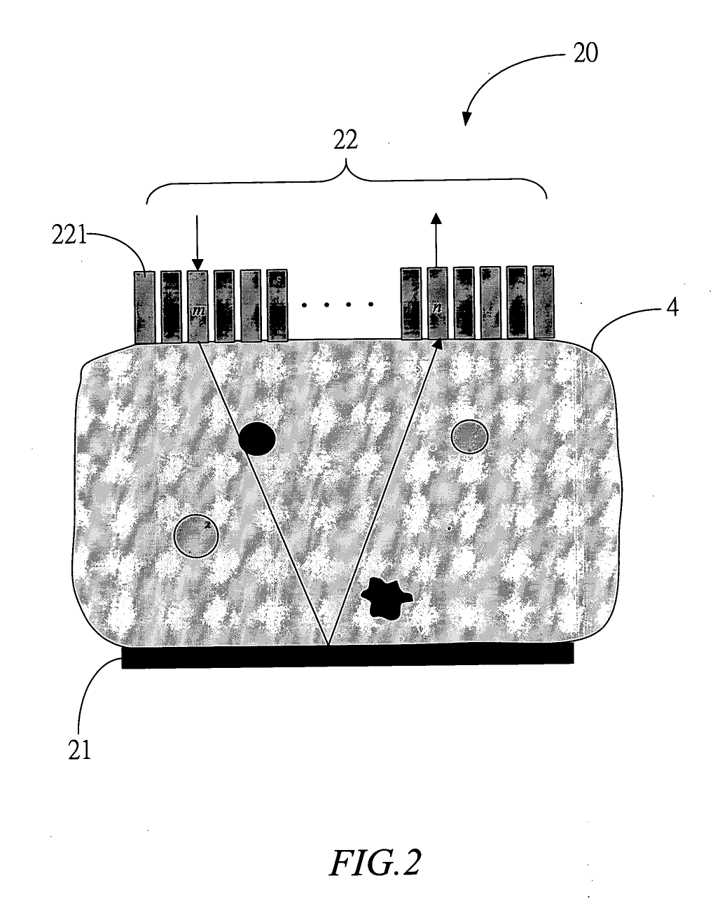

FIG. 2 is a diagram of imaging equipment 20 of the invention. Different from the conventional computed tomography apparatus, the imaging equipment 20 applies a reflector 21 reflecting the transmitted signal from every channel 221 of a transducer array 22. It enables the system to get the echoes from the reflector for all transmit/receive combinations, and then to reconstruct the sound velocities and the attenuation coefficients of different tissues of an image object 4 using the echoes.

Comparing with the conventional method, the invention does not need to rotate the ultrasound transducers, and is compatible with the conventional B-mode imaging apparatus. Despite the advantages, the sound velocities and the attenuation coefficients cannot be accurately estimated with the conventional computed tomography reconstruction methods due to the fact that that measurement angles span less than 180 degrees (i.e., incomplete data). The invention applies proper reconstruction constraints derived from the B-mode image obtained with the same imaging apparatus to assist in accurately estimating image parameters such as sound velocity and attenuation coefficient.

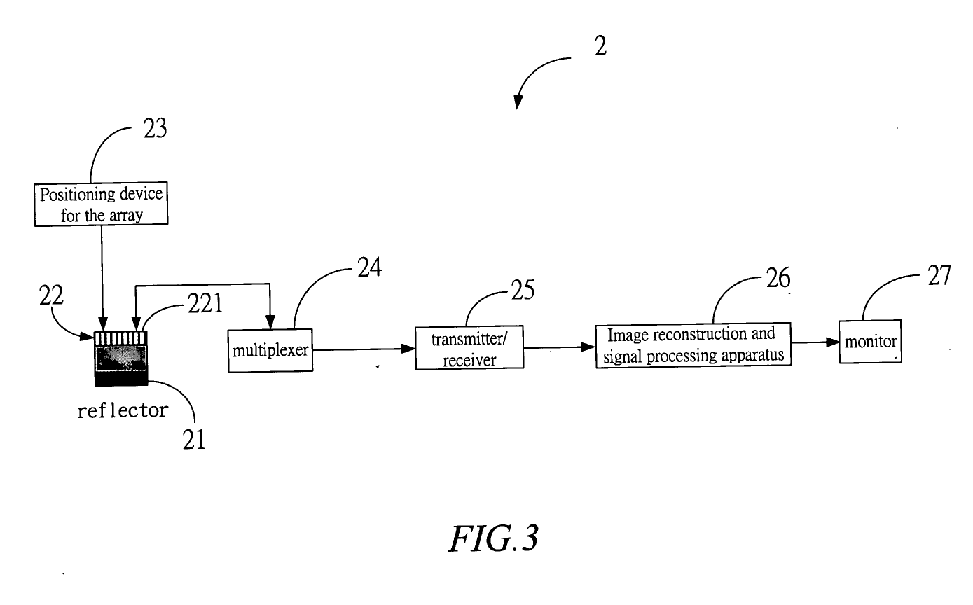

FIG. 3 is a schematic diagram of the imaging apparatus in the invention. The ultrasound imaging apparatus 2 of the invention comprises:

a transducer array 22 located on one side the image object with a plurality of channels 221 which can transmit and receive independently and can be turned on or off by a multiplexer 22. Furthermore, the scanning may be electronic or mechanical, and its geometry may be linear or nonlinear;

a positioning device 23 for the array, which is used to keep good contact between the transducer array 22 and the image object, extend imaging area, obtain a 3-dimentional image, and compress the object to perform elasticity imaging;

a reflector 21 located on the other side of the image object opposite to the transducer array 22, which can effectively reflect ultrasonic wave incident from any angle and may be planar or nonplanar. Furthermore, the reflector 21 may be replaced by another receive array;

a multiplexer 24, which can independently turn on or turn off any channel of the transducer array 221;

a transmitter/receiver 25, which transmits or receives signals via the channels 221 of the transducer array 22;

an image reconstruction and signal processing apparatus 26, which receives the detected signal from the transmitter/receiver 25, and estimates the sound velocities and the attenuation coefficients of the object using the time-of-flight data and the attenuation data extracted from the receive signals, together with the segmentation information of the object derived from the corresponding B-mode image; and

a monitor 27, which displays the results of the image reconstruction and signal processing apparatus 26.

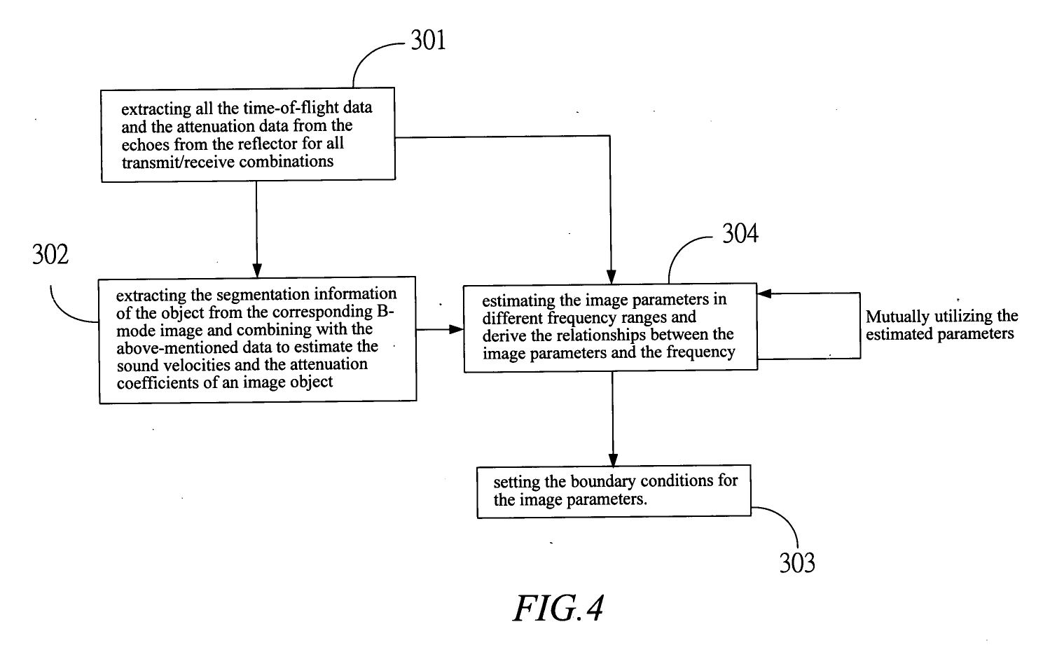

FIG. 4 illustrates an implementation method of the invention. It includes steps of:

301, i.e. extracting all the time-of-flight data and the attenuation data from the echoes for all transmit/receive combinations;

302, i.e. extracting the segmentation information of the object from the B-mode image and combining with the above-mentioned data to estimate the sound velocities and attenuation coefficients of an object;

303, i.e. setting the boundary conditions for the image parameters such that the parameters can be estimated in different frequency ranges; and

304, i.e. deriving the relationships between the image parameters and the frequency, e.g. slope.

The ultrasound imaging apparatus and method is superior to conventional technologies in the following aspects:

1. The ultrasound imaging apparatus and method of the invention can acquire the ultrasound B-mode image, the sound velocities, the attenuation coefficients, and the elastic properties of an object, and then use them to identify the tissue types (e.g., to detect the breast tumor).

2. The ultrasound imaging apparatus and method of the invention applies an ultrasound transducer array for transmitting and receiving ultrasound signals, without rotating the array or the object, to obtain a B-mode image, the sound velocities, and the attenuation coefficients. It also can be extended to obtaining other image parameters, e.g. elastic modulus, to provide further information of the object.

3. The ultrasound imaging apparatus and method of the invention applies the information extracted from the B-mode image of the object to effectively improve the estimation accuracy of the sound velocities and the attenuation coefficients without using other imaging apparatus.

While the present invention has been described in conjunction with the preferred embodiments thereof, it is evident that many alternatives, modifications and variations will be apparent to those skilled in the art. Accordingly, it is intended to embrace all such alternatives, modifications and variations that fall within the spirit and scope thereof as set forth in the appended claims.

It can tell that the invention is innovative and improved relative to conventional arts. It would be thankful if the application can be granted without misgivings.

Many changes and modifications in the above-described embodiment of the invention can, of course, be carried out without departing from the scope thereof. Accordingly, to promote the progress in science and the useful arts, the invention is disclosed and is intended to be limited only by the scope of the appended claims.

### REFERENCE

1. S. A. Goss, R. L. Johnston, and F. Dunn, “Comprehensive compilation of empirical ultrasonic properties of mammalian tissues,” J. Acoust. Soc. Amer., vol. 64, no. 2, pp. 423-457, 1978.

2. A. C. Kak and M. Slaney, Principles of Computerized Tomographic Imaging, New York: the Institute of Electrical and Electronics Engineers, Inc., 1988.

3. B. J. Smith and R. R. Adhami, “Medical imaging. Computerized tomography,” IEEE Potentials, vol. 17, no. 5, pp. 9-12, December 1998/January 1999.

4. M. Krueger, A. Pesavento, and H. Ermert, “A modified time-of-flight tomography concept for ultrasonic breast imaging,” in Proc. of IEEE Ultrason. Symp., 1996, pp. 1381-1385.

5. M. Krueger, V. Burow, K. M. Hiltawsky, and H. Ermert, “Limited angle ultrasonic transmission tomography of the compressed female breast,” in Proc. of IEEE Ultrason. Symp., 1998, pp. 1345-1348.

What is claimed is:

1. An ultrasound imaging apparatus comprising:
a transducer array located on one side of an image object with a plurality of channels which can transmit and receive independently, and can be turned on or off by a multiplexer;
a positioning device for the array, which is used to keep good contact between the transducer array and the image object, extend imaging area, obtain a 3-dimentional image, and compress the object to perform elasticity imaging;
a reflector located on the other side of the image object opposite to the transducer array, which can effectively reflect ultrasonic wave incident from any angle;
a multiplexer, which can independently turn on or turn off any channel of the transducer array;
a transmitter/receiver, which transmits or receives detected signals via the channels of the transducer array;
an image reconstruction and signal processing apparatus, which receives the detected signal from the transmitter/receiver, and estimates the sound velocities and the attenuation coefficients of the objects using the time-of-flight data and the attenuation data extracted from the receive signal, together with the segmentation information of the object derived from the B-mode image; and
a monitor, which displays the results of the image reconstruction and signal processing apparatus.

2. The ultrasound imaging apparatus of claim 1, wherein the scanning of the transducer array may be electronic or mechanical, and the geometry of the array may be linear or nonlinear.

3. The ultrasound imaging apparatus of claim 1, wherein the reflector may be planar or nonplanar.

4. The ultrasound imaging apparatus of claim 1, wherein the reflector may be replaced by another receive array.

5. An ultrasound imaging method, which includes steps of:
extracting all the time-of-flight data and the attenuation data from the echoes from the reflector for all transmit/receive combinations;
extracting the segmentation information of the object from the B-mode image and combining with the above-mentioned data to estimate the sound velocities and the attenuation coefficients of an image object; and
setting the boundary conditions for the image parameters.

6. The ultrasound imaging method of claim 5, which estimates the image parameters in different frequency ranges and derive the relationships between the image parameters and the frequency.

7. The ultrasound imaging method of claim 5 or claim 6, wherein the image parameters include sound velocity, attenuation coefficient, and elastic modulus.
